(19) United States
(12) Patent Application Publication
(10) Pub. No.: US 2023/0355125 A1
(43) Pub. Date: Nov. 9, 2023

(54) THROMBOSIS DIAGNOSING GUIDE WIRE

(71) Applicant: HeMo Bioengineering Pvt. Ltd., San Jose, CA (US)

(72) Inventors: Jack Wang, Singapore (SG); Udayan Patel, San Jose, CA (US); Samantha Ng Huey Xin, Singapore (SG); Goh Pei Wen, Singapore (SG); Tee Tay Zoe, Singapore (SG); Kok Hui Ping, Singapore (SG); John Wong, Singapore (SG); Lukas Lee, San Jose, CA (US)

(21) Appl. No.: 18/108,452

(22) Filed: Feb. 10, 2023

Related U.S. Application Data
(60) Provisional application No. 63/309,326, filed on Feb. 11, 2022.

(51) Int. Cl. A61B 5/0538; A61B 5/00; A61B 5/02; A61B 5/145

(52) U.S. Cl. CPC A61B 5/0538; A61B 5/721; A61B 5/6851; A61B 5/6847; A61B 5/7214; A61B 5/02007; A61B 5/14503

(57) ABSTRACT

The present disclosure relates to a medical device in the form of a medical guide wire. The medical guide wire can be used in delivering catheters to treatment sites within human vasculature, and can also be configured to simultaneously allow the user to determine the nature of the blockage within human vasculature. The medical guide wire can be used to determine the extent of organization of thrombus by sensing the electrical resistivity across a blockage. The medical guide wire can include a hollow core through which at least two electrical leads run along the partial or full length of the medical guide wire from proximal to distal end of the medical guide wire. The medical guide wire can include two or more sensors at its distal portion and the sensors are separated from each other.

### REFERENCE TO RELATED APPLICATIONS

The present application claims priority on U.S. Provisional Application Ser. No. 63/309,326 filed Feb. 11, 2022, which is fully incorporated herein by reference.

### THE FIELD OF DISCLOSURE

The present disclosure relates to a medical device in the form of a medical guide wire and a method for using the medical device for diagnosing a thrombus and cancerous cells and providing an optimal treatment. The medical guide wire can be used in delivering catheters to a treatment site within the animal or human vasculature, and can also be configured to simultaneously allow the user to determine the nature of a blockage within the animal or human vasculature. The medical guide wire can be used to determine the extent of organization of thrombus by sensing the electrical resistivity across a blockage, and/or be used to detect the presence of cancerous cells in the blood flowing through the blood vessel. The medical guide wire can include a hollow core through which at least two electrical leads run along the partial or full length of the medical guide wire from proximal to distal end of the medical guide wire. The medical guide wire can include two or more sensors at its distal end region, and wherein the two or more sensors are separated from each other. During use of the medical guide wire, a low current can be induced into one or more first sensors. A signal from the thrombus within the animal or human vasculature can thereafter be detected by one or more of the second sensors. This detected signal can be converted to an impedance value or some other value to identify the thrombus type within the animal or human vasculature. The medical guide wire can be used to assist a physician in choosing the appropriate thrombus-removing technique and obtaining optimum patient recovery. The received signals can also or alternatively be used to detect the presence of cancerous cells in the blood that flow past the medical guide wire.

### BACKGROUND OF DISCLOSURE

Stenosis in a blood vessel is caused by the formation of plaque or thrombus in a blood vessel. The modern means of relieving the stenosis is by angioplasty in which dilatation of the blood vessel is driven by a balloon catheter. However, not all stenosed vessels can be treated by angioplasty, especially in the neural arteries. Most blockages that cause stroke are due to thrombus formation at the location of the blockage or thrombus emboli originating from other parts of the arterial system. It is well known that the majority of the thrombus-containing lesions are not angiographically pre-identified as thrombotic; the thrombus can be overshadowed by the necrotic core of a plaque. While the thrombus is understated, the calcium is overly identified. In either case, the best solution to may be the removal of the thrombus by thrombectomy or simply suction.

The thrombus is formed typically due to plaque rupture. The fresh thrombus is soft and over time gets more organized and fibrous. The fresh thrombus is relatively easier to remove than the aged organized thrombus. It has been reported by physicians that there is a difference in the feel when crossing the thrombus with a guide wire, thus somewhat indicating to the physician the extent of the thrombus. Based on the extent to which the thrombus has organized, an appropriate treatment is applied. In general, a soft thrombus is removed easily by using an aspiration catheter while an organized thrombus may require maceration of the thrombus followed by suction or by using a stent retriever. When the thrombus is extremely organized and hardened, more drastic means, such as rotational or vibrational thrombectomy devices, are employed.

In more recent medical device developments, there have been combination devices with atherectomy and aspiration or combination of stent retriever with aspiration. This approach is effective in many cases where there may exist a thrombus with a range of organization extending radially from wall to center of the vessel. However, without knowing the exact nature of the thrombus, it is very likely that treatment with various devices may prove to be extremely aggressive, in which case adverse effects, such as dissections or an embolic event, can occur. On the other hand, a less aggressive treatment may leave a partially stenosed vessel.

Blood thrombus formation process changes the blood structure and composition. The structural changes during blood coagulation induce alterations in the blood conductivity. A decrease in blood conductivity with evolution of the clotting process measuring and blood conductivity has been observed. The relative blood conductivity decreases with increasing hematocrit levels. The absolute change of conductivity (Δσ) equals the difference of blood conductivity at complete clotting (σc) and blood conductivity at the beginning of clotting process (σs) as indicated by the following formula: Δσ=|σc−σs|.

Other than blood thrombus, the speed of cancerous cells spreading can be identified from the difference in conductivity. Poorly differentiated cancer cells grow and spread more slowly than well differentiated cancer cells. It has been observed that poorly differentiated cancer cells have higher conductivity than normal cells.

There is an unfulfilled need for diagnosing a thrombus and cancerous cells and providing an optimal treatment. The medical device in accordance with the present disclosure addresses the shortfalls of current devices and fulfills a medical need by providing to the caregiver the means by which to obtain accurate identification of the nature of stenosis and subsequent careful selection of the treatment modality.

### SUMMARY OF DISCLOSURE

In accordance with one non-limiting aspect of the present disclosure, there is provided a medical device in the form of a medical guide wire and a method for using the medical device for diagnosing a thrombus and cancerous cells and providing an optimal treatment. The medical guide wire can be used in delivering catheters to a treatment site within the animal or human vasculature, and can also be configured to simultaneously allow the user to determine the nature of a blockage within the animal or human vasculature. The medical guide wire can be used to determine the extent of organization of thrombus by sensing the electrical resistivity across a blockage, and/or be used to detect the presence of cancerous cells in the blood flowing through the blood vessel. The medical guide wire can include a hollow core through which at least two electrical leads run along the partial or full length of the medical guide wire from proximal to distal end of the medical guide wire. The medical guide wire can include two or more sensors at its distal end region, and wherein the two or more sensors are separated from each other. During use of the medical guide wire, a low current can be induced into one or more first sensors. A signal from the thrombus within the animal or human vasculature can thereafter be detected by one or more of the second sensors. This detected signal can be converted to an impedance value or some other value to identify the thrombus type within the animal or human vasculature. The medical guide wire can be used to assist a physician in choosing the appropriate thrombus-removing technique and obtaining optimum patient recovery. The received signals can also or alternatively be used to detect the presence of cancerous cells in the blood that flow past the medical guide wire.

In accordance with another non-limiting aspect of the present disclosure, the medical guide wire is directed, but not limited to, the determining of one or more properties of a stenosis on a blood vessel treatment of a diagnosis of stenosis in a blood vessel by determining the impedance in the electrical current across the stenosis.

In accordance with another non-limiting aspect of the present disclosure, the medical guide wire is directed, but not limited to, the detection of cancerous cells in the blood flowing through a blood vessel by determining the impedance in the electrical current in the blood vessel.

In accordance with another non-limiting aspect of the present disclosure, the medical guide wire includes a body (e.g., tubular body, semi-tubular body, solid body, etc.) that extends partially or fully along the longitudinal length of the medical guide wire, and at least one sensor. Generally the medical guide wire includes two or more sensors at or near the distal end of the medical guide wire. Generally, the one or more sensors are located closer to the distal end of the medical guide wire than to the proximal end of the medical guide wire. In one non-limiting embodiment, the one or more sensors are located a distance from the distal end of the medical guide wire of 0-20% (and all values and ranges therebetween) of the longitudinal length of the body of the medical guide wire, and typically the one or more sensors are located a distance from the distal end of the medical guide wire of 0.5-10% of the longitudinal length of the body of the medical guide wire. In one non-limiting arrangement, the one or more sensors are spaced from a distal end of the medical guide wire. In another non-limiting arrangement, all of the sensors are spaced from the distal end of the medical guide wire. In another non-limiting arrangement two or more sensors are spaced different distances from the distal end of the medical guide wire. When the medical guide wire includes two or more sensors, the sensors are generally located at a fixed distance apart along the longitudinal length of the medical guide wire; however, this is not required. The sensors are located at or near the distal end of the medical guide wire. If one sensor is located at or near the distal end of the medical guide wire, another sensor is generally located on the body of the medical guide wire and spaced along the longitudinal axis of the medical guide wire distally from the sensor that is at or near the distal end. As can be appreciated, two or more of the sensors can be spaced from the distal end of the medical guide wire, but still be located near the distal end of the medical guide wire. The one or more sensors can be placed such that 1) at least one of the sensor is at least partially exposed to the outer surface of the medical guide wire, and/or 2) at least one of the sensors is positioned in the interior of the medical guidewire. In one non-limiting arrangement, all of the sensors are at least partially located on the exterior surface of the medical guide wire. In another non-limiting arrangement, all of the sensors are located within the medical guide wire and spaced from an exterior surface of the medical guide wire. In another non-limiting arrangement, at least one of the sensors is at least partially located on the exterior surface of the medical guide wire, and at least one of the sensors is located within the medical guide wire and spaced from an exterior surface of the medical guide wire. The material that forms the sensors is non-limiting. In another non-limiting embodiment, the one or more sensors are formed of or include a metal such as, but not limited to, gold, platinum, titanium, etc. The size and shape of the one or more sensors is non-limiting. In another non-limiting embodiment, the one or more sensors have a top surface that has a round shape, square-shape, oval shape, polygonal shape, obround shape, etc., and has a maximum dimension of at least 10 μm (e.g., 10-350 μm and all values and ranges therebetween). In another non-limiting embodiment, the shape of one or more of the sensors can be a band shape that partially of fully encircles the outer surface of the body of the medical guide wire. In one non-limiting arrangement, the band has a maximum width of at least 10 μm (e.g., 10-350 μm and all values and ranges therebetween) and typically 100-240 μm. In another non-limiting embodiment, the one or more sensors can have a circular shape, a square shape, a triangular shape, an oval shape, a polygonal shape, etc., and the shape of the one or more sensors has a maximum cross-sectional dimension on the outer surface of the medical guide wire of at least 10 μm (e.g., 10-350 μm and all values and ranges therebetween) and typically 100-240 μm. As can be appreciated, the one or more sensors can be larger (e.g., up to the full length of the medical device). Generally, when two or more sensors are used, at least two of the sensors have the same shape, size, and/or are formed of the same material. The distance apart of two or more sensors on the medical guide wire is non-limiting. In one non-limiting embodiment, the distances apart of two or more sensors on the medical guide wire are about at least 100 μm (e.g., 100-800 μm and all values and ranges therebetween) and typically 300-400 μm. As can be appreciated, when one or more sensors are used on other types of the medical devices (e.g., stent, balloon guide, catheter, etc.), the same or similar parameters of the sensors can optionally be used.

In accordance with another non-limiting aspect of the present disclosure, the medical guide wire optionally includes a hollow body or tube (e.g., hollow tubular body, etc.) that extends partially or fully along the longitudinal length of the medical guide wire. In one non-limiting embodiment, the longitudinal length of the cavity in the tube is at least 10% (e.g., 10-100% and all values and ranges therebetween) of the longitudinal length of the tube. At least one, and generally at least two electrical wires are located within the cavity of the tube and extend from a location at or near the proximal end of the medical guide wire to each of the sensors located at or near the distal end of the medical guide wire. At or near the proximal end of the medical guide wire, the one or more electrical wires can be connected to an electrical circuit source that is capable of sending current through at least one of the electrical wires and to the one or more sensors. The electric current source or some other detecting device can be used to detect the current from one or more of the sensors via one or more of the electrical wires. In one non-limiting arrangement, one electrical wire is used to send a current from the electric current source to one or more sensors, and another electrical wire is used to send a current from one or more other sensors to the electric current source or some other detecting device.

In accordance with another non-limiting aspect of the present disclosure, the medical guide wire optionally includes a body or tube having a distal portion that includes one or more cut-out pattern portions to form a flexible tip region of the medical guide wire to enhance the flexibility of the distal region of the medical guide wire. Such a flexible tip region facilitates in the insertion of the medical guide wire into the vasculature of a patient. The shape of each of the cut-out patterns on the flexible tip region is non-limiting. In one non-limiting embodiment, the flexible tip portion includes a plurality of cut-out pattern portions. In another non-limiting embodiment, the shape and/or size of two or more cut-out portions is the same. In another non-limiting embodiment, the cut-out portions on the distal portion of the medical guide wire constitute at least 10% (e.g., 10-75% and all values and ranges therebetween) of the surface area of the medical guide wire at such distal end portion. In another non-limiting embodiment, the one or more cut-out patterns are located distally of the sensors of the medical guide wire. In another non-limiting embodiment, the one or more cut-out patterns extend from a region on the medical guide wire that is proximal to the sensor to a region on the medical guide wire that is distal to the sensors of the medical guide wire. In another non-limiting embodiment, one or more of the cut-out patterns are formed in the body of the medical guide wire, and the depth of the one or more cut-out patterns extends partially or fully through the wall of the body or tube.

In accordance with another non-limiting aspect of the present disclosure, the medical guide wire optionally includes a body or tube having a distal end portion that is tapered from the proximal end of the distal end portion towards the distal end of the distal end portion. The tapered section is optionally coated with a jacket of polymeric material that optionally contains radiopaque material and/or particles. The tapered section facilitates in providing flexibility to the medical guide wire as it is inserted in and through a body passageway (e.g., blood vessel, etc.). As such, a flexible tip (when used) can facilitate in the insertion of the medical guide wire into the vasculature of a patient.

In accordance with another non-limiting aspect of the present disclosure, the shape and placement of the sensors on the medical guide wire are selected to obtain accurate electrical signal processing of the signal detected from the vasculature. Contact impedance varies with different sensor sizes and surface area in contact with the thrombus. Sensors with larger surface area can detect a larger surface area of the thrombus. This allows for measurement of the overall thrombus impedance. Conversely, a sensor with small surface area allows for more precise point measurements along the thrombus. In terms of sensor placement, the spacing between sensors affects the penetration of current and area to which the current travels, thus affecting the signal detected.

In accordance with another non-limiting aspect of the present disclosure, one non-limiting advantage of using the medical guide wire in accordance with the present disclosure is to increase first-pass thrombectomy success rate. The medical guide wire can be used to accurately identify thrombus composition based on the measured impedance value captured from the sensor when in direct contact with the thrombus. This technique of identifying thrombus composition via a medical guide wire is believed to be novel. Currently, there is no effective way to identify the stiffness of thrombus in the vasculature. Therefore, physicians in the past have only been able to use the method of trial and error for thrombus removal. By using the medical guide wire in accordance with the present disclosure to measure thrombus composition and/or stiffness, a physician is able to measure thrombus impedance and thereby categorize the properties of the thrombus more efficiently and accurately. The stiffness of the thrombus can be deciphered from thrombus impedance to determine a treatment plan. Hence, effectively categorizing thrombus will significantly increase the success rate of first-pass thrombectomy.

In accordance with another non-limiting aspect of the present disclosure, one or more of the sensors are positioned on the outer surface of the body or tube of the medical guide wire. In one non-limiting embodiment, all of the sensors are positioned on the outer surface of the body or tube of the medical guide wire. In one non-limiting embodiment, the outer surface of the tube includes a recess for one or more of the sensors such that when the sensor is connected to the outer surface of the body or tube, the top of the sensor is flush with or recessed from the top surface of the body or tube; however, this is not required. In another non-limiting embodiment, the body or tube includes wire openings for one or all of the sensors that are connected to the outer surface of the body or tube so that the wire that is connected to the sensor can pass from the sensor, through the wire opening, and into the interior cavity of the body or tube. Such arrangement can be used to partially or fully prevent the wire that is connected to the sensor from being exposed to the outer surface of the body or tube. In another non-limiting arrangement, the wire opening is located in one or more of the recess for the sensors.

In accordance with another non-limiting aspect of the present disclosure, there is provided a noise sensor to reduce or cancel noise that is received from the other sensors on the guide wire. The noise sensor can optionally be connected to an electronic circuit (e.g., differential amplifier, amplifier noise canceller, etc.) to facilitate in the cancellation of noise. For example, the noise senor can function as a differential probe that is used as a reference to the signals received form the sensors. The noise sensor can optionally be used to look at small signals in the presence of large DC offsets. The DC offset shift can be many folds higher than the sensing signal amplitudes. The use of a differential probe has more common mode rejection which can extract the small signal among high-shifted signal. By doing this, a clearer signal (not shifted and less noisy signal) can be obtained. For example, a single signal from one of the sensors and the noise sensor can be amplified before being converted into a single-ended signal. After being amplified, the two signals can be passed through into a converter from the differential to a single-ended signal. During this step, the single-ended signal can be filtered from noise and offset with its high common mode rejection capability. The output signal of this circuitry can then be fed into a frequency generator. As can be appreciated, the frequency generator also can include an add-on module to convert from differential input to single-ended output (e.g., Differential Electrometer Amplifier (DEA)).

In accordance with another non-limiting aspect of the present disclosure, there is provided an optional substrate that can be partially positioned in the tube of the guide wire. The sensors can be optionally positioned on the substrate. As such, the substrate can be used as a securing surface for the one or more sensors. The one or more sensors can be secured to the substrate in a variety of arrangements (e.g., adhesive, melted connection, solder, friction fit, hook and loop fastener, etc.). Wires and/or contact pads can optionally be positioned on the surface and/or in the substrate. As can be appreciated, the one or more sensors, wires and/or contact pads can be printed (e.g., 3D printed, photo-etched, etc.) on the substrate. The substrate (when used) is generally formed of a low electrically conducting or non-electrically conducting material (e.g., polymers, plastics, foam material, rubber, ceramic, composite material, etc.). The substrate can have a cross-sectional shape that is the same or similar to the cross-sectional shape of the cavity of the tube. The length of the substrate is generally about 0.1-25% (and all values and ranges therebetween) of the longitudinal length of the tube. Generally, the substrate is positioned from the distal end of the tube a distance of 0.01 to 25% a longitudinal length of the tube (and all values and ranges therebetween). The substrate can be at least partially secured in the cavity of the body or tube by a variety of arrangements (e.g., adhesive, melted connection, solder, friction fit, hook and loop fastener, etc.). The substrate can optionally include a cut or shaved region so that when the sensors are connected to the cut or shaved region, the top surface of the sensor is flush with or recessed from the outer surface of the body or tube when the substrate is secured in the cavity of the body or tube. When the substrate is used, the body or tube can optionally include a sensor cut-out area. The cut-out area allows the sensors on the substrate to have direct contact with a thrombus when the guide wire is inserted into the vasculature. The cut-out area is generally located at the distal portion of the body or tube. The location of the cut-out portion is generally spaced from the distal end of the body or tube about 0.1-25% (and all values and ranges therebetween) of the longitudinal length of the tube. The length of the cut-out portion is generally about 0.1-25% (and all values and ranges therebetween) of the longitudinal length of the tube. The longitudinal length of the substrate is generally the same or greater than a longitudinal length of the cut-out portion. The width of the substrate is generally the same or greater than width of the cut-out portion.

In accordance with one non-limiting object of the present disclosure, there is provided a medical diagnostic system that includes a) a medical device; b) an electrical assembly at the distal portion of the medical device to transmit and receive an electrical signal of the diagnosis site; and c) an analyzer that optionally includes user interface to analyze the collected data and process and/or reflect the results.

In accordance with another non-limiting object of the present disclosure, the medical guide wire can be used to accurately identify how poorly differentiated the cancer cells are based on the measured impedance value captured from the sensor when in direct contact with the tumour.

In accordance with another non-limiting object of the present disclosure, there is provided a medical diagnostic system wherein the electrical assembly comprises one or more sensors.

In accordance with another non-limiting object of the present disclosure, there is provided a medical diagnostic system wherein the sensors can have alternative configurations in terms of shape, quantity, placement, and size.

In accordance with another non-limiting object of the present disclosure, there is provided a medical diagnostic system wherein the sensors can be optionally printed or otherwise formed (e.g., 3D printed, photo etched, plated, adhesively connected, molded to the substrate, etc.) on a flexible substrate.

In accordance with another non-limiting object of the present disclosure, there is provided a medical diagnostic system wherein the sensors of the medical device can be detached, and other sensors can be attached to capture more information for diagnosis. These sensors can obtain data on, but are not limited to, shear force, pressure, impedance, dissipation, stress, and flow.

In accordance with another non-limiting object of the present disclosure, there is provided a medical diagnostic system that further includes conductive metals and/or contact pads that are optionally printed or otherwise formed (e.g., 3D printed, photo etched, plated, adhesively connected, molded to the substrate, etc.) onto the flexible substrate with the sensors.

In accordance with another non-limiting object of the present disclosure, there is provided a medical diagnostic system wherein the contact pad connects to electrical assembly in the medical device.

In accordance with another non-limiting object of the present disclosure, there is provided a medical diagnostic system wherein the electrical assembly includes one or more electrical wires.

In accordance with another non-limiting object of the present disclosure, there is provided a medical diagnostic system wherein the electrical assembly is electrically coupled to the impedance analyzer and optionally includes a user interface to form a closed-loop circuit.

In accordance with another non-limiting object of the present disclosure, there is provided a medical diagnostic system wherein the electrical assembly is connected to alternating current.

In accordance with another non-limiting object of the present disclosure, there is provided a medical diagnostic system wherein the medical device is a guide wire, the guide wire optionally includes a hollow proximal section that transits into a solid core distal section; and a solid core distal section of the guide wire optionally features cuts and gaps to enhance the flexibility of the guide wire to reach tortuous sites; and one or more electrical sensors are placed near the distal end of the guide wire; and electrical wires are routed through the hollow guide wire that are configured to connect the electrical sensors to an analyzer; and an optional solid core support wire that is routed through the hollow guide wire to increase the pushability of the guide wire.

In accordance with another non-limiting object of the present disclosure, there is provided a medical diagnostic system including a tissue-sensor interface located at the distal hollow section of the guide wire, the tissue-sensor interface includes a cut-out area to allow the sensors to have direct contact with the tissue.

In accordance with another non-limiting object of the present disclosure, there is provided a medical diagnostic system wherein the guide wire is introduced into a vessel pathway of a body, and navigated along the vessel until the thrombus site and the electrical sensors are in contact with the tissue so as to acquire the signals for the analysis of the tissue composition.

In accordance with another non-limiting object of the present disclosure, there is provided a medical diagnostic system wherein the solid core support wire optionally replaces a single electrical wire that connects the electrical sensor to the analyzer.

In accordance with another non-limiting object of the present disclosure, there is provided a medical diagnostic system wherein the medical device is a stent.

In accordance with another non-limiting object of the present disclosure, there is provided a medical diagnostic system wherein the medical device is a balloon guide wire.

In accordance with another non-limiting object of the present disclosure, there is provided a medical diagnostic system wherein the medical device is a catheter.

In accordance with another non-limiting object of the present disclosure, there is the provision of a medical diagnostic system comprising a) a medical device that is configured to be inserted into a blood vessel of an animal or human; the medical device includes a distal portion having a distal end and a proximal portion having a proximal end; b) an electrical assembly at least partially located at the distal portion of the medical device; the electrical assembly includes first and second sensors located at the distal portion of the medical device; the electrical assembly configured to transmit and receive one or more electrical signals at a diagnostic site when the distal portion of the medical device is located at the diagnosis site; and c) an impedance analyzer configured to analyze one or more the signals received from the electrical assembly.

In accordance with another non-limiting object of the present disclosure, there is the provision of a medical diagnostic system wherein the medical device is selected from the group consisting of a guide wire, a balloon guide wire, stent retriever or a catheter.

In accordance with another non-limiting object of the present disclosure, there is the provision of a medical diagnostic system wherein the medical device includes a radiopaque marker positioned a) adjacent to one or the first or second sensors, b) between the first and second sensor, or c) distally to the first and second sensors.

In accordance with another non-limiting object of the present disclosure, there is the provision of a medical diagnostic system wherein the electrical assembly includes a noise reduction sensor positioned on the distal portion of the medical device; the noise reduction sensor spaced from the first and second sensors; the noise reduction sensor positioned a) proximally to the first and second sensors, b) between the first and second sensors, or c) distally to the first and second sensors.

In accordance with another non-limiting object of the present disclosure, there is the provision of a medical diagnostic system that further includes an electronic circuit selected from the group consisting of a differential amplifier, and an amplifier noise canceller.

In accordance with another non-limiting object of the present disclosure, there is the provision of a medical diagnostic system wherein the first or second sensors are exposed an outer surface of the medical device so that at least a portion of the first or second sensors can contact an inner wall of a blood vessel or material located on the inner wall of the blood vessel when the distal portion of the medical device is positioned the diagnosis site.

In accordance with another non-limiting object of the present disclosure, there is the provision of a medical diagnostic system wherein the medical device includes a tube; an outer surface of the tube includes first and second sensor recesses; the first sensor recess configured to at least partially receive the first sensor; the second sensor recess configured to at least partially receive the second sensor; a top surface of the first sensor is flush with or recessed from an outer surface of the tube when the first sensor is positioned in the first sensor recess; a top surface of the second sensor is flush with or recessed from an outer surface of the tube when the second sensor is positioned in the second sensor recess.

In accordance with another non-limiting object of the present disclosure, there is the provision of a medical diagnostic system wherein the medical device includes a tube that includes an internal cavity that extends at least 60% of a longitudinal length of the tube; the electrical assembly including a first wire electrically connected to the first sensor and a second wire electrically connected to the second sensor; the first and second wires positioned in at least 50% of a longitudinal length of the cavity of the tube.

In accordance with another non-limiting object of the present disclosure, there is the provision of a medical diagnostic system wherein a majority of a longitudinal length the first and second wires that are located in the cavity are not connected to the cavity.

In accordance with another non-limiting object of the present disclosure, there is the provision of a medical diagnostic system wherein a distal portion of the tube includes one or more recesses or cut-out portions that are configured to increase a flexibility of the distal portion of the tube; the one or more recesses or cut-out portions positioned a) proximally to the first and second sensors, b) between the first and second sensors, or c) distally to the first and second sensors.

In accordance with another non-limiting object of the present disclosure, there is the provision of a medical diagnostic system wherein the medical device includes a rod that is positioned in the cavity of the tube and extends from a proximal end of the tube to a distal portion of the tube; a majority of a length of the rod is not connected to the cavity.

In accordance with another non-limiting object of the present disclosure, there is the provision of a medical diagnostic system wherein the medical device includes a tube substrate that is positioned at least partially in the cavity of the tube; the first and second sensors are connected to a top surface of the tube substrate; the tube substrate formed of a different material from the tube.

In accordance with another non-limiting object of the present disclosure, there is the provision of a medical diagnostic system wherein the tube substrate includes a shaved region that reduces a cross-sectional area of the shaved region as comparted to regions of the tube substrate located proximal and/or distal to the shaved region; the first and second sensors at least partially positioned on the shaved region.

In accordance with another non-limiting object of the present disclosure, there is the provision of a medical diagnostic system wherein the tube substrate has a longitudinal length of 1-25% a longitudinal length of the tube; the tube substrate has a cross-sectional shaped that is the same or similar to a cross-sectional shape of the cavity of the tube.

In accordance with another non-limiting object of the present disclosure, there is the provision of a medical diagnostic system wherein the tube includes a cut-out area positioned at the distal portion of the tube; the tube substrate positioned in the cavity and oriented related to the cut-out areas such that a top surface of the first and second sensors that are connected to the top surface of the tube substrate are exposed an outer surface of the medical device.

In accordance with another non-limiting object of the present disclosure, there is the provision of a medical diagnostic system wherein the medical device is a balloon guide wire; the balloon guide wire includes a tube and an inflatable balloon; an outer surface of the balloon includes the first and second sensors.

In accordance with another non-limiting object of the present disclosure, there is the provision of a medical diagnostic system wherein the medical device is a stent retriever; the stent retriever includes a plurality of interconnected wires; the first and second sensors connected to an outer surface of the interconnected wires.

In accordance with another non-limiting object of the present disclosure, there is the provision of a medical diagnostic system wherein a distal portion of the medical device includes a tapered region.

In accordance with another non-limiting object of the present disclosure, there is the provision of a medical diagnostic system wherein the tapered region of the distal portion is coated with a radiopaque material.

In accordance with another non-limiting object of the present disclosure, there is the provision of a method for using the medical diagnostic system to a) obtain one or more properties of a thrombus in a blood vessel, and/or b) identify an existence or presence of cancer cells flowing within the blood vessel.

In accordance with another non-limiting object of the present disclosure, there is the provision of a medical device that is configured to be inserted into a blood vessel of an animal or human; the medical device includes a tube and an electrical assembly; the tube includes an internal cavity that extends at least 60% of a longitudinal length of the tube; the tube has a distal portion having a distal end and a proximal portion having a proximal end; the electrical assembly at least partially located at the distal portion of the medical device; the electrical assembly includes first and second sensors; the electrical assembly includes a first wire electrically connected to the first sensor and a second wire electrically connected to the second sensor; the first and second wires positioned in at least 50% of a longitudinal length of the cavity of the tube; the first and second sensors are located at the distal portion of the medical device; the electrical assembly configured to transmit and receive one or more electrical signals at a diagnostic site when the distal portion of the medical device is located at the diagnosis site.

These and other advantages of the present disclosure will become more apparent to those skilled in the art from a review of the description of the preferred embodiment and claims.

### BRIEF DESCRIPTION OF THE DRAWINGS

Reference may now be made to the drawings, which illustrate various embodiments that the disclosure may take in physical form and in certain parts and arrangements of parts wherein:

FIG. 1A is a cross-sectional view of a portion of an exemplary embodiment of a diagnostic medical guide wire with sensors in accordance with the present disclosure;

FIG. 1B is a top view of a portion of the diagnostic medical guide wire as shown in FIG. 1A;

FIG. 1C is a cross-sectional view of a portion of a portion of a modified diagnostic medical guide wire as shown in FIG. 1A;

FIG. 1D is a n enlarged sectional view of the diagnostic medical guide wire as shown in FIG. 1A which illustrates sensors on a portion of the medical guide wire;

FIG. 2A is a cross-sectional view of a portion of a portion of the diagnostic medical guide wire as shown in FIG. 1A which illustrates the medical guide wire connected to a current source and/or current sensing source;

FIG. 2B is a cross-sectional view of a portion of the diagnostic medical guide wire as shown in FIG. 1A which illustrates the medical guide wire disconnected to a current source and/or current sensing source;

FIG. 3 is a cross-sectional view of a portion of the diagnostic medical guide wire as shown in FIG. 1A which illustrates a non-limiting electric impedance measurement system for the medical guide wire;

FIG. 4A is a portion of an electrical circuit that can be used in the electric impedance measurement system for the medical guide wire;

FIG. 4B is a portion of an electrical circuit that can be used in the electric impedance measurement system for the medical guide wire;

FIG. 4C is a portion of an electrical circuit that can be used in the electric impedance measurement system for the medical guide wire;

FIG. 4D is a portion of an electrical circuit that can be used in the electric impedance measurement system for the medical guide wire;

FIG. 5A is a n enlarged sectional view of the diagnostic medical guide wire as shown in FIG. 1A which illustrates the medical guide wire deployed inside a blood vessel, and wherein two sensors on the medical guide wire lay across a stenosis in the blood vessel;

FIG. 5B is a n alternative enlarged sectional view of the diagnostic medical guide wire as shown in FIG. 1A which illustrates the medical guide wire deployed inside a blood vessel, and wherein two sensors (e.g., ring electrodes) on the medical guide wire lay across a stenosis in the blood vessel;

FIG. 6A is a n alternative enlarged sectional view of the diagnostic medical guide wire as shown in FIG. 1A which illustrates the diagnostic guide wire without solid core support;

FIG. 6B is another alternative enlarged sectional view of the diagnostic medical guide wire as shown in FIG. 1A which illustrates the diagnostic guide wire without solid core support;

FIG. 7 is a n enlarged isometric view of the diagnostic medical guide wire as shown in FIG. 1A which illustrates one of the electrical wire replaced by a solid core support wire inside the diagnostic medical guide wire;

FIG. 8 is another embodiment of the present disclosure illustrating one or more sensors located on a stent retriever;

FIG. 9A is another embodiment of the present disclosure illustrating a cross-sectional view of a balloon guide wire that includes one or more sensors;

FIG. 9B is an enlarged isometric view of the balloon guide wire of FIG. 9A illustrating one or more sensors on a balloon guide wire;

FIG. 10A is an enlarged partial isometric view of a medical guide wire that includes four sensors on the medical guide wire;

FIG. 10B is an enlarged partial isometric view of a medical guide wire that includes six sensors on the medical guide wire;

FIG. 10C is another enlarged partial isometric view of a medical guide wire that includes six sensors on the medical guide wire;

FIG. 10D is a top view of the medical guide wire of FIG. 10C;

FIG. 11 is another enlarged partial isometric view of a medical guide wire that includes a tapered tip, and can optionally be flexible, and the tapered tip optionally includes a coating that can optionally include a radiopaque material;

FIG. 12 is a portion of an electrical circuit that can be used in the electric impedance measurement system with a differential amplifier for the medical guide wire; and

FIG. 13 is a portion of an electrical circuit that can be used in the electric impedance measurement system with a differential amplifier for the medical guide wire.

### DETAILED DESCRIPTION OF NON-LIMITED EMBODIMENTS

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the present disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g., “about 2 to about 4” also discloses the range “from 2 to 4.” Generally, the terms “about” and “approximately” may refer to plus or minus 10% of the indicated number.

Referring now to the Figures, there is illustrated non-limiting embodiments of the medical guide wire in accordance with the present disclosure that can be used to effectively categorize thrombus to significantly increase the success rate of first-pass thrombectomy.

A thrombus can block or narrow an artery which can lead to the brain thereby resulting in ischemic stroke. The blood thrombus is typically formed due to plaque rupture. The main composition of thrombi are fibrin and erythrocyte. Depending on the ratio, thrombi can be classified into fibrin-rich thrombus and erythrocyte-rich thrombus. The stiffness of thrombi corresponds to its composition.

In addition to plaque rupture, rarer causes of ischemic stroke can occur due to embolism. This phenomenon occurs when a blood thrombus forms elsewhere in the body, breaks off as an embolus, and travels through the bloodstream to the brain. These types of thrombi are usually calcified and form what is known as calcified cerebral emboli (CCE).

In general, blood thrombi do not have a fixed shape. The shape of the blood thrombi change according to the shape of the blood vessels. The ability to mold to the shape of the blood vessels also depend on the stiffness of the thrombus itself.

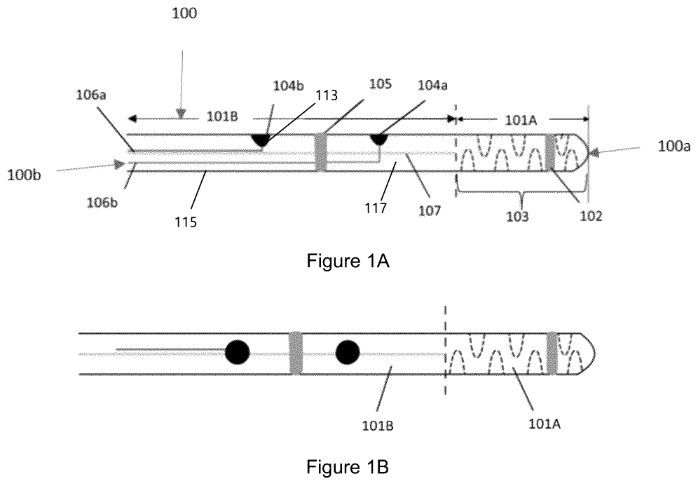

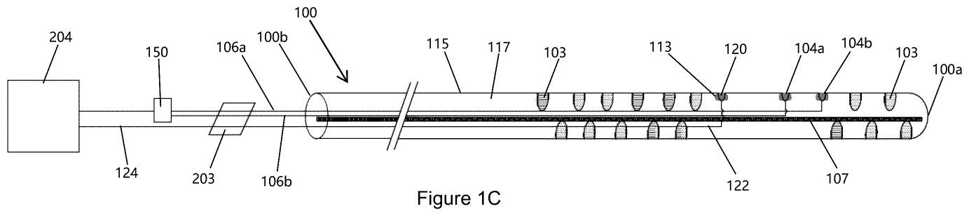

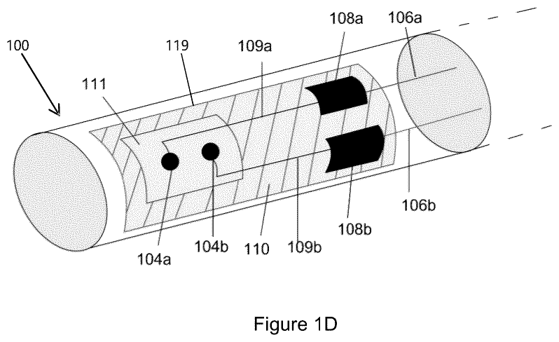

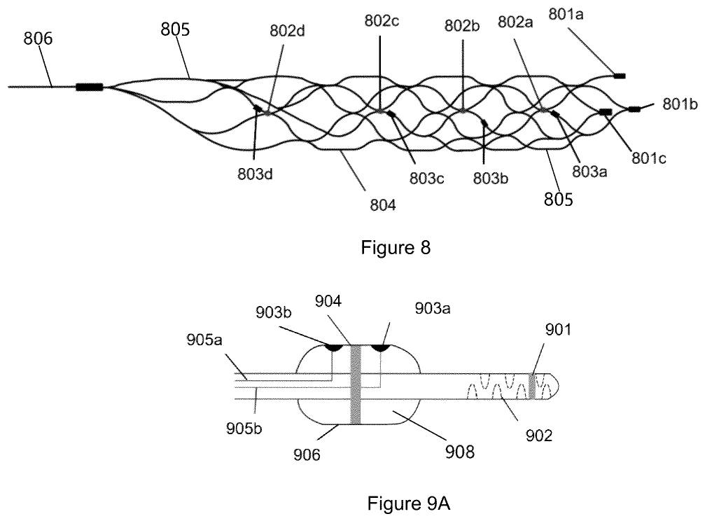

Referring now to FIGS. 1A, 1B and 1C, there is illustrated an exemplary embodiment of a medical device in the form of a diagnostic guide wire 100 that includes a tube 115 and sensors 104a, 104b. As can be appreciated, other types of medical devices in accordance with the present disclosure can be used with sensors to detect/obtain properties of a thrombus 301 in a vasculature. (See FIGS. 8, 9A, and 9B.

Referring again to FIGS. 1A, 1B and 1C, guide wire 100 includes a tube 115 that includes a cavity 117 that extends the partial or full longitudinal length of the 115. In one non-limiting arrangement, cavity 117 extends 3-100% (and all values and ranges therebetween) of the longitudinal length of tube 115. Generally, the cross-sectional shape and size of the cavity remains constant along the longitudinal length of cavity 117; however, this is not required. In one non-limiting configuration, cavity 117 has a generally circular cross-sectional shape; however, other shapes can be used (e.g., polygonal, oval, etc.). The tube can be formed of a variety of materials (e.g., metal, plastic, composite material, etc.). In one non-limiting configuration, tube 115 is formed of a metal such as nitinol, SS, CoCr, Re alloy, molybdenum alloy, etc.

Referring again to FIGS. 1A, 1B and 1C, the distal tip 100a of tube 115 of guide wire 100 is generally partially or fully closed; however, this is not required. Distal tip 100a of tube 115 can be closed by variety of arrangements such as, but not limited to a) at least partially forming distal tip 100a with a solid metal, b) soldering or welding the end of the tube, or c) crushing or clamping closed the end or end portion of the tube. Distal tip 100a can optional be smoothed and/or rounded and/or tapered.

The distal section 101A of tube 115 of the wire 100 can optionally include cuts and gaps 103 that are configured to increase the flexibility of distal section 101A of the tube of the diagnostic guide wire. In one non-limiting arrangement, the distal section of the guide wire can optionally be formed of a solid core with distal cuts and gaps 103. In another non-limiting arrangement, the distal section of the guide wire can partially or fully include cavity 117, and the distal cuts and gaps 103 may or may not partially or fully penetrate the wall of tube 115. The distal section of the tube can be formed of the same or different material from other portions of the tube. The length of the distal end of the tube that includes the optional distal cuts and gaps 103 is generally at least 0.1% (e.g., 0.1-20% and all values and ranges therebetween) of the longitudinal length of the tube.

Referring again to FIGS. 1A, 1B and 1C, 101B represents the region of the tube located proximal to distal section 101A. The composition of tube 115 is non-limiting (e.g., SS, CoCr, Re alloy, Mo alloy, nitinol, etc.). The body portion of the diagnostic guide wire as represented by 101B is optionally a hollow tube. Optionally positioned in cavity 117 of tube 115 of the diagnostic guide wire are two or more electrical wires such as 106a, 106b. The hollow tube can partially or fully enclose the one or more electrical wires. The composition of the electrical wire is non-limiting (e.g., copper, aluminum, etc.). Wires 106a, 106b (illustrated as being laid within the tubular body) extend to and generally beyond proximal end 100b of the tube and also extend to each of sensors 104a, 104b or are electrically connected to an electrical wire connection of the sensors. In one non-limiting arrangement, less than 25% (e.g., 0.00001-25% and all values and ranges therebetween) of the longitudinal length of wires 106a, 106b is connected to the tube.

As illustrated in FIGS. 1A and 1B, sensors 104a, 104b can optionally be located distally to the optional distal cuts and gaps 103. However, it can be appreciated that when distal cuts and gaps 103 are used, distal cuts and gaps 103 can extend both proximally and distally to sensors 104a, 104b as illustrated in FIG. 1C. The sensors can be formed of a variety of materials (e.g., polymer, metal [e.g. copper, aluminum, platinum, titanium, gold, etc.).

In one non-limiting arrangement, sensors 104a, 104b can be placed on the tube such that the sensors are at least partially exposed to the outer surface of the tube of the guide wire. However, it can be appreciated that one or more or all of the sensors can be located fully within cavity 117 of tube 115. The sensors can be connected to the tube by a variety of arrangements (e.g., adhesive, solder, weld, melted connection, clamp, magnet, etc.).

When one or more or all of the sensors are located at least partially on an outer surface of the tube, the tube can optionally include a sensor recess 113 for one or more or all of the sensors so that the top surface of the sensor is located flush with or slightly recessed from the outer surface of the tube.

As illustrated in FIGS. 1A, 1B and 1C, sensors 104a, 104b are spaced from distal section 101A of the diagnostic guide wire; however, it will be appreciated that one or both of the sensors can be partially or fully located in distal section 101A of the diagnostic guide wire. When one or both sensors are located on the distal region of the tube, the one or both sensors are generally positioned closer to distal tip 100a of the guide wire than to proximal end 100b of the guide wire. In one non-limiting arrangement, the sensors 104a, 104b are spaced form one another a distance of at least 10 μm (e.g., 10-1000 μm and all values and ranges therebetween).

As illustrated in FIGS. 1A, 1B and 1C, a core support wire 107 is optionally placed in the hollow tube to increase the pushability of the guide wire to facilitate in the ability of the guide wire to be inserted in the vasculature. Core support wire 107 can be a solid or hollow wire. Core support wire 107 can be formed for a variety of materials (e.g., metal, plastic, ceramic, composite material, etc.). In one non-limiting configuration, core support wire 107 can be formed of a metal such as nitinol, SS, CoCr, rhenium alloy, molybdenum alloy, etc. The cross-sectional area of core support wire 107 along 50-100% (and all values and ranges therebetween) of the longitudinal length of the cavity of the tube is generally less than the cross-sectional area of the cavity of the tube. In one non-limiting configuration, the distal region of core support wire 107 is connected to the tube. As illustrated in FIGS. 1A and 1B, the distal tip of core support wire 107 is connected to distal section 101A of the tube; however, it can be appreciated that the core support wire 107 can be connected to other or additional portions of the tube. When the distal section 101A of the tube is a solid material, the distal tip of core support wire 107 can be connected to the distal end of distal section 101A; however, this is not required. As illustrated in FIG. 1C, the core support wire 107 can extend to or closely adjacent to distal tip 100a of tube 115. The connection arrangement between distal section 101A of tube and core support wire 107 is non-limiting (e.g., friction connection, clamp connection, solder, adhesive, etc.). In one non-limiting arrangement, less than 25% (e.g., 0.00001-25% and all values and ranges therebetween) of the longitudinal length of core support wire 107 is connected to the tube. In another non-limiting arrangement, only the front end or front end portion of core support wire 107 is connected to the tube.

As illustrated in FIGS. 1A and 1B1, one or more marker bands 102, 105 can be optionally placed on the tube of the guide wire to allow visualization under angiography to facilitate in the proper positioning of the guide wire in the vasculature. A distal tip marker band 102 (when used) helps physicians to detect the location of the tip or distal region of guide wire when inserting the guide wire in the vasculature. A sensor marker band 105 (when used) helps physicians locate the position of sensors 104a, 104b in the vasculature when inserting the guide wire in the vasculature. As can be appreciated, only one marker band can be used or more than two marker bands can be used. As illustrated in FIGS. 1A and 1B, one marker band is located between two of the sensor; however, it can be appreciated that the marker bands can be located in other locations (e.g., one each side one each sensor, on each side of a pair or set of sensors, distally of the sensors, at the location of one or more sensors, etc.). The position of the one or more marker bands in the guide wire is non-limiting. Generally, the one or more marker bands are positioned on the distal region of the guide wire. Generally, the marker bands are positioned from the distal end of the tube a distance of 0.01 to 25% a longitudinal length of the tube (and all values and ranges therebetween). The material used to form the marker bands is non-limiting. Generally, the marker material is a different material from the material used to form the tube. As can be appreciated, one or more marker bands can optionally be included on the guide wire illustrated in FIG. 1C (e.g., a marker band arrangement that is similar to FIGS. 1A, etc.).

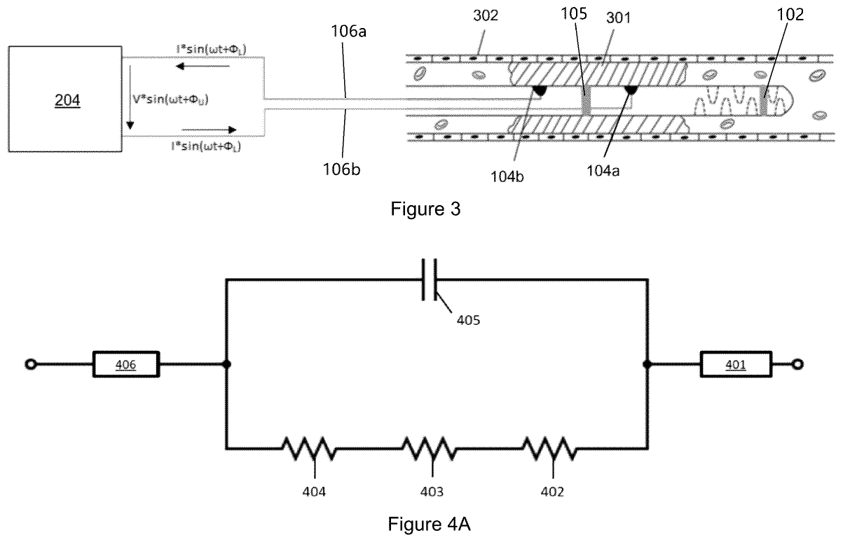

Referring now to FIG. 3, the guide wire can optionally include a noise sensor 120 reduce or cancel noise that is received from sensors 104a, 104b. Noise sensor 120 is connected to an electrical wire 122. Electrical wire 122, similar to electrical wires such as 106a, 106b, extends through the cavity of the tube to proximal end 100b of guide wire 100. Electrical wire 122 can be the same or similar to wires such as 106a, 106b as discussed above. In one non-limiting arrangement, less than 25% (e.g., 0.00001-25% and all values and ranges therebetween) of the longitudinal length of wire 122 is connected to the tube. Generally, noise sensor 120 is at least partially positioned on the outer surface of the tube; however, it can be appreciated that noise sensor 120 can be positioned in the cavity of the tube. When noise sensor 120 is positioned on the outer surface of the tube, noise sensor 120 can optionally be at least partially positioned in sensor recess 113, and the top surface of noise sensor 120 can optionally be flush with or recessed from the top surface of the tube. As illustrated in FIG. 1C, noise sensor 120 is positioned distally of sensors 104a, 104b. Generally, noise sensor 120 is generally positioned within 0.01-20 cm (and all values and ranges therebetween). Noise sensor 120 is generally positioned at the distal portion of the tube (e.g., positioned from the distal end of the tube a distance of 0.01 to 15% a longitudinal length of the tube and all values and ranges therebetween). Noise sensor 120 can be the same or similar to sensors 104a, 104b.

FIGS. 1C and 1D illustrate sensors 104a, 104b positioned on an optional substrate 110. The substrate is used as a securing surface for the one or more sensors. The one or more sensors can be secured to the substrate in a variety of arrangements (e.g., adhesive, melted connection, solder, friction fit, hook and loop fastener, etc.). The conductive metal 109a, 109b (e.g., bare wires, coated and/or insulated wires, etc.) can be optionally positioned on the outer surface of substrate 110 and/or be positioned within the substrate 110. Contact pads 108a, 108b can be optionally positioned on the outer surface of substrate 110 and/or be positioned within substrate 110. Contact pads 108a, 108b form a connection between conductive metal 109a, 109b of sensors 104a, 104b and electrical wires 106a, 106b. As can be appreciated, contact pads 108a, 108b can be eliminated and some other type of electrical connection between conductive metal 109a, 109b of sensors 104a, 104b and electrical wires 106a, 106b can be used (e.g., solder connection, wire coupler, etc.). Substrate 110 (when used) is generally formed of a low electrically-conducting or non-electrically-conducting material (e.g., polymers, plastics, foam material, rubber, ceramic, composite material, etc.). In one non-limiting embodiment, the substrate includes a cross-sectional shape that is the same or similar to the cross-sectional shape of the cavity of the tube. In non-limiting configuration, the substrate has a generally cylindrical shape. The length of the substrate (when used) is generally about 0.1-25% (and all values and ranges therebetween) of the longitudinal length of the tube, and typically the length of the substrate is about 0.1-5% of the longitudinal length of the tube. Generally, the substrate is positioned from the distal end of the tube a distance of 0.01 to 25% a longitudinal length of the tube (and all values and ranges therebetween), and typically the substrate is positioned from the distal end of the tube a distance of 0.01 to 5% a longitudinal length of the tube. The substrate can be at least partially secured in the cavity of the tube by a variety of arrangements (e.g., adhesive, melted connection, solder, friction fit, hook and loop fastener, etc.).

Substrate 110 can optionally include a cut or shaved region 111 so that when the sensors are connected to cut or shaved region 111, the top surface of the sensor are flushed with or recessed from the outer surface of the tube when the substrate is secured in the cavity of the tube.

In one non-limiting configuration, substrate 110 has a length of at least 50 μm (e.g., 50-5000 μm and all values and ranges therebetween), and a maximum width or diameter of at least 20 μm (e.g., 20-500 μm and all values and ranges therebetween). In another non-limiting configuration, the sensors positioned on substrate 110 are spaced apart at least 10 μm (e.g., 10-1000 μm and all values and ranges therebetween). The sensors can be positioned along the central axis of the substrate; however, this is not required. As illustrated in FIG. 1C, the sensors are spaced from the proximal and distal ends of the substrate. Contact pads 108a, 108b are illustrated as being spaced from one another and are spaced from the proximal and distal ends of the substrate. Contact pads 108a, 108b are also illustrated as spaced from the cut or shaved region 111; however, this is not required. Contact pads 108a, 108b and/or conductive metals 109a, 109b (e.g., wires, etc.) can be positioned on the outer surface of substrate 110 or can be partially or fully positioned in the interior of the substrate.

Referring now to FIG. 1D, the tube can optionally include a sensor cut-out area 119. The length of the optional cut-out area 119 (when used) is generally about 0.1-25% (and all values and ranges therebetween) of the longitudinal length of the tube, and typically the length of cut-out area 119 is about 0.1-5% of the longitudinal length of the tube. In one non-limiting arrangement, when the guide wire includes substrate 110, the length of the substrate is generally the same or greater than the length of cut-out area 119 to facilitate in the securing of the substrate in the tube. Generally, cut-out area 119 is positioned from the distal end of the tube a distance of 0.01 to 25% a longitudinal length of the tube (and all values and ranges therebetween), and typically the cut-out area is positioned from the distal end of the tube a distance of 0.01 to 5% a longitudinal length of the tube. The maximum perimeter length of cut-out area 119 is generally no more than 90% of the perimeter of the tube (e.g., 1-90% and all values and ranges therebetween), and typically maximum perimeter length of cut-out portion is generally 5-50% of the perimeter of the tube.

Cut-out area 119 allows sensors 104a, 104b, that are positioned in the substrate, to have near or direct contact with thrombus 301 when the guide wire is inserted into the vasculature to obtain a precise and accurate impedance measurement of the thrombus as illustrated in FIG. 3. Substrate 110 can be customized by varying the placement of sensors 104a, 104b on the substrate. The sensors can optionally be connected to a conductive metal 109a, 109b at one end, and the other end of the conductive metal can be connected to contact pads 108a, 108b. Contact pads 108a, 108b can then be used to electrically connect the conductive material to electrical wires 106a, 106b. As can be appreciated, electrical wires 106a, 106b can be directly connected to the sensors. The sensors, conductive material, and/or contact pads can optionally be connected to the inner surface of the guide wire by use of an adhesive (e.g., epoxy resin, etc.). As can be appreciated, the sensors, conductive material, and/or contact pads can optionally be connected to the inner surface of the guide wire by other arrangements (e.g., solder, mechanical connection, etc.).

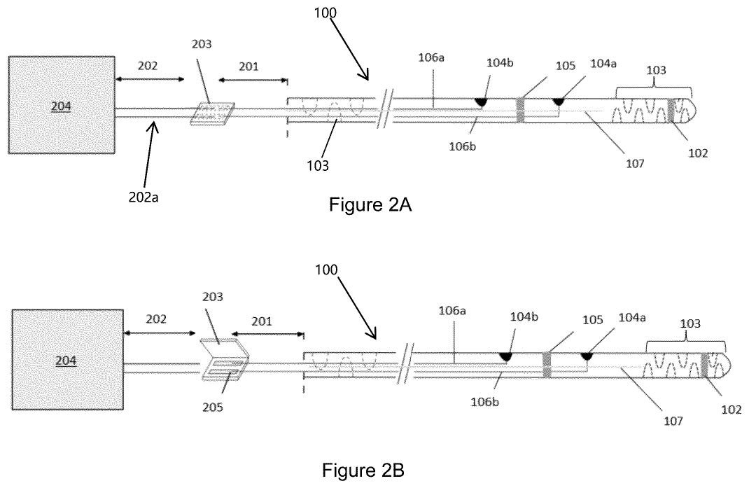

As illustrated in FIGS. 1C, 2A, and 2B, electrical wires 106a, 106b are connected to sensors 104a, 104b via contact pad 108a, 108b and conductive metal 109a, 109b. Wires 106a, 106b are then run within the cavity of the tube of the guide wire toward proximal end 100b of guide wire 100. Free end portion 201 of electrical wires 106a, 106b that is located at or near proximal end 100b of the tube of the guide wire can optionally be attached to wire connector 203. Use of wire connector 203 allows for wires 106a, 106b to be detached and reconnected easily to free end portion 201 of electrical wires 106a, 106b to free end portion 202 of wires 202a that are connected to impedance analyzer 204. Likewise, the use of wire connector 203 allows for wire 122 to be detached and reconnected easily to free end portion 201 of electrical wire 122 to free end portion of wire 124 that is connected to impedance analyzer 204 as illustrated in FIG. 1C. As can be appreciated, free end portion 201 of electrical wires 106a, 106b and/or the free end portion of wire 124 can be directly connected to impedance analyzer 204. Free end portion 201, electrical wire 106a, 106b, and/or wire 122 can be designed such that the length would be as short as possible to ensure ease of use of the guide wire. Upon determining the ideal mechanical thrombectomy method, free end portion 201 of electrical wire 106a, 106b and/or wire 122 can be detached from wire connector 203 to facilitate insertion of the guide wire in the vasculature; however, this is not required. Once the guide wire is inserted into the desired location in the vasculature, free end portion 201 of electrical wire 106a, 106b and/or wire 122 can be again attached to wire connector 203 so that the guide wire is electrically connected to impedance analyzer 204.

Platinum, titanium, and gold are recommended materials for a portion or all sensors 104a, 104b, 120; however, the sensors are not limited to these materials. The materials of conductive metal 109a, 109b and contact pad 108a, 108b can include and/or be generally the same material used to form sensors 104a, 104b; however, this is not required. The optional substrate 110 (which can optionally be flexible) can be partially or fully formed of a polymer (e.g., polyimide, parylene-C, etc.). The one or more electrical wires 106a, 106b, 122 can be formed of copper (e.g., copper C101, copper C110, etc.); however, other conductive materials can be used. The wires and/or conductive metal and/or contact pads can be partially or fully coated with a low or non-electrically conductive material (e.g., plastic, polymer, etc.).

Table 1 illustrates non-limiting examples of materials that can be used for the various components of the medical device; however, it will be appreciated that other comparable materials can be used. Table 1 also illustrates non-limiting parameters of the components on the medical device; however, it will be appreciated that other parameters can be used.

TABLE 1
Material and Description of Component

| Components | | Description |
|---|---|---|
| Sensors | Material | Platinum or Titanium or Gold |
| | Shape | Round or Rectangular |
| | Spacing between sensors | 300-400 μm |
| | Size | 100-240 μm |
| | Quantity | 2 or more sensors |
| | Connection | Electrically Conductive |
| | | Epoxy Adhesives |

| | |
|---|---|
| Substrate | Polyamide or Parylene-C |
| Conductive metal | Platinum or Titanium or Gold |
| Contact pad | Platinum or Titanium or Gold |
| Electrical wire | Copper (C101, 99.99 wt. % Cu) or |
| | Copper (C110, 99.9 wt. % Cu) |

Referring now to FIGS. 1C, 2A, and 2B, sensors 104a, 104b, once the guide wire is properly positioned in the vasculature, are in direct contact with thrombus 301 as illustrated in FIG. 3, the signal from the current that interacts with thrombus 301 is sent to an impedance analyzer 204 through electrical wire 106a and/or 106b from sensors 104a and/or 104b.

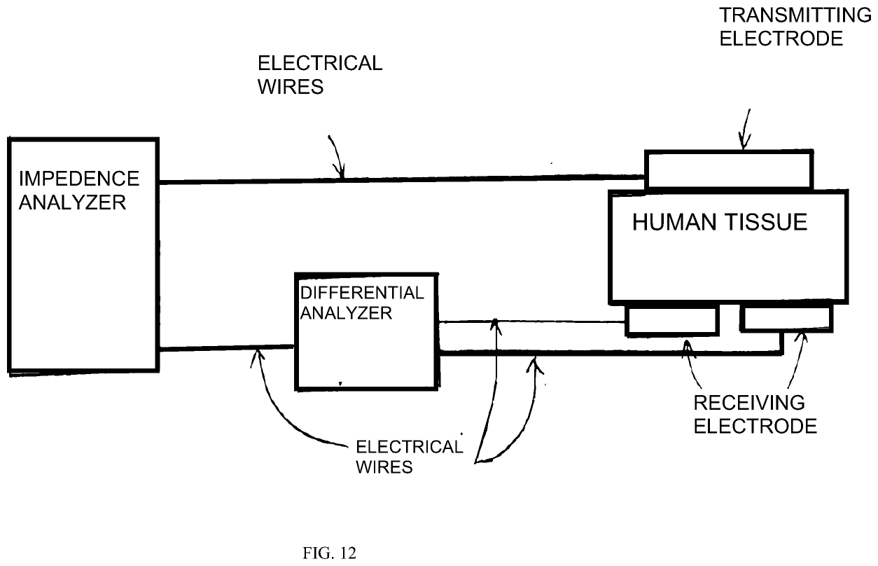

Referring now to FIGS. 1C and 12, when the guide wire includes a noise sensor 12, one or both signals from sensors 104a and/or 104b can be processed through electronic circuit 150 (e.g., differential amplifier, amplifier noise canceller, current source, current sensing source, etc.) to facilitate in the cancellation of noise. The signal from sensors 120 and 104a and/or 104b can be further processed in impedance analyzer 204. As illustrated in FIGS. 1C and 12, the medical guide wire is connected to a differential amplifier 150 and current source and/or current sensing source that can be used in the electric impedance measurement system with differential amplifier 150 for the medical guide wire. The input signal from analyzer 204 is introduced into the circuit via the single-end electrical wire to transmitting sensor 104b. Transmitting sensor 104b transmits the signal through the tissue and is captured by receiving sensor 104a. The signals from receiving sensor 104a and also optionally the input sigma are amplified by differential amplifier 150. At the time of analysis, the analyzed signal can be optionally filtered from noise and/or offset with its high common mode rejection capability. After signal amplification, the processed signal is converted into a single ended signal, and the converted output signal is transmitted into analyzer 204.

To allow for clearer illustration, FIGS. 1A, 1B, 1C, 2A, 2B and 3 illustrate a guide wire having a two electrical sensors 104a, 104b configuration; however, it will be appreciated that the guide wire can include more than two sensors.

FIG. 2A illustrates the connection of free end portion 201 of electrical wires 106a, 106b to ends 202 of wires 202a to form a closed configuration between the guide wire and impedance analyzer 204. When wire connector 203 is closed, the circuit forms a closed loop and impedance analyzer 204 is able to read the impedance of the thrombus that was detected by the sensors on the guide wire. The wire connector can optionally include one or more conductive pads 205 that are used to allow electrical signals to pass between electrical wires 106a, 106b and wires 202a. A similar connection arrangement is illustrated FIG. 3 which includes the addition of wire 122.

FIG. 2B illustrates electrical wires 106a, 106b and wires 202 in the open configuration. When wire connector 203 is opened, electrical wires 106a, 106b can be electrically disconnected from impedance analyzer 204 and the impedance of the thrombus cannot be measured. As can be appreciated, such disconnection arrangement can be used for wire 122 illustrated in FIG. 1C. The ability to allow the guide wire to be disconnected from impedance analyzer 204 ensures that the mechanical thrombectomy equipment can pass over the guide wire with ease.

The signal from sensors 104a, 104b can be captured, converted, and analyzed using impedance analyzer 204 and the information from impedance analyzer 204 can be used to aid with the determination of treatment method for the thrombosis in the vasculature. An optional electronic circuit 150 (e.g., differential amplifier, amplifier noise canceller etc.) in combination with the use of noise sensor 120 can be used to facilitate in the cancellation of noise to improve the determination of treatment method for the thrombosis in the vasculature.

FIG. 3 illustrates a schematic drawing of electric impedance measurement system. An AC power source is used in impedance analyzer 204. The current direction illustrated in FIG. 3 is solely for illustrative purpose. It should be appreciated that the current flow direction changes with alternating current source used. The signal that is transmitted through electrical wires 106a, 106b generally has a maximum allowable voltage drop of no more than about 10% (e.g., 0-10% and all values and ranges therebetween); however, this is not required. Impedance analyzer 204 is used to measure the composition and/or other properties (e.g., density, size, etc.) of thrombus 301. The parameters of sensors such as sensor size, spacing, and/or placement on the guide wire, as well as the contact pad 108a, 108b area can be controlled to obtain desired signal readings related to thrombus 301. These parameters can be selected to ensure low signal to noise ratio (SNR) and a more accurate analysis of thrombus 301.

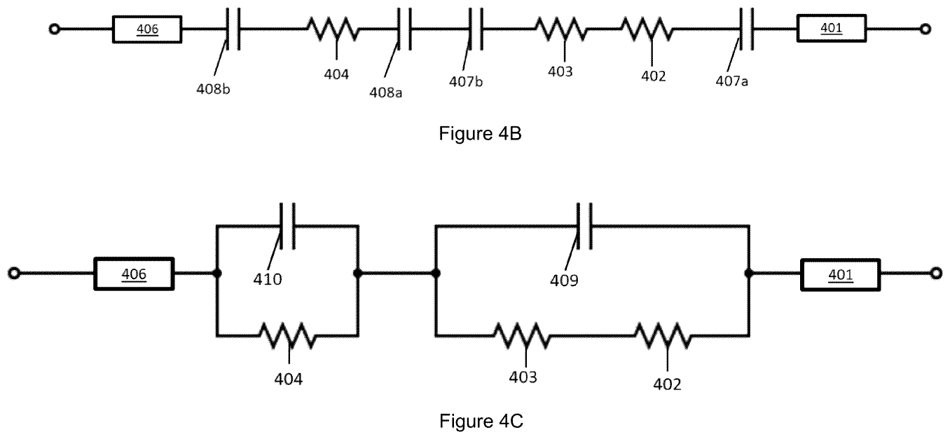

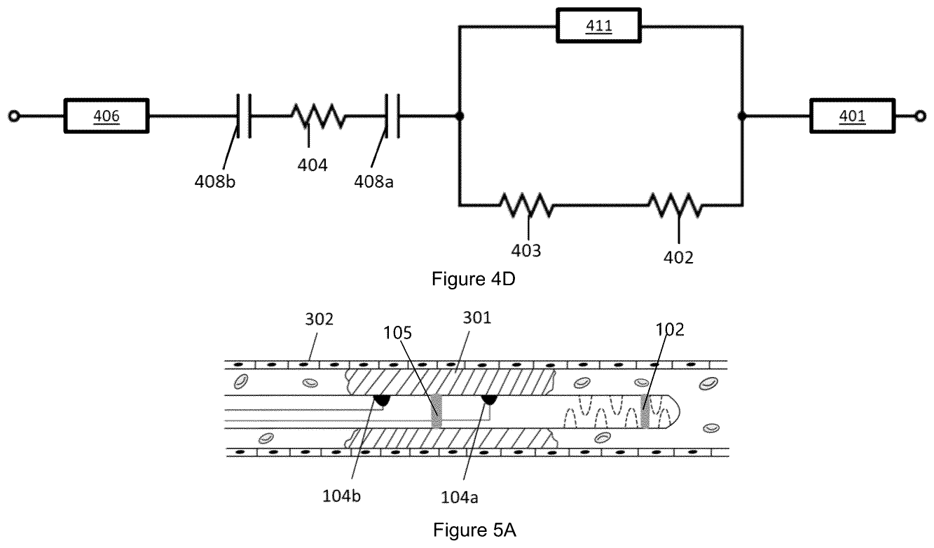

FIGS. 4A. 4B, 4C and 4D illustrate non-limiting possible circuit models for two-sensor configurations on the guide wire. Four possible circuits can be used to simulate the impedance of thrombus 301 in a vasculature with varying degrees of electrical signaling. The impedance of measured tissue is modeled as a combination of resistors 402, 403, 404 and capacitors 405, 407a, 407b, 408a, 408b, 409, 410 in parallel and series. In order to illustrate the non-ideal double layer capacitance in real-life situation at the sensor-tissue interface, both working sensor and counter sensor are considered as constant phase elements (CPE) 401, 406 as represented by the following formula:

Where

$${Z}_{CPE} = \frac{1}{{{Y}_{CPE} ( j ω )}^{α}}$$

Z_{CPE} denotes the impedance of working sensor and counter sensor
Y_{CPE} denotes the nominal capacitance value
j=\sqrt{−1}
ω denotes the angular frequency of the alternating current
α denotes a constant between 0 and 1

FIG. 4B includes the use of two capacitors 408a, 408b to simulate the double membrane of cells while a CPE 411 is added to the model to simulate non-ideal double layer capacitance of tissues in real-life situations, with the total impedance as represented by the following formula:

$${Z}_{T} = {Z}_{CPE} + \frac{2}{ω C} + {R}_{1} + \frac{{z}_{{CPE}_{1}} ( {R}_{2} + {R}_{3} )}{{Z}_{{CPE}_{1}} + ( {R}_{2} + {R}_{3} )}$$

Where
Z_{T} denotes the total impedance of the model
Z_{CPE} denotes the impedance of working sensor and counter sensor
Z_{CPE}_{₁} denotes the impedance of tissues
ω denotes the angular frequency of the alternating current
C denotes the capacitance
R denotes the resistance of the elements in the model

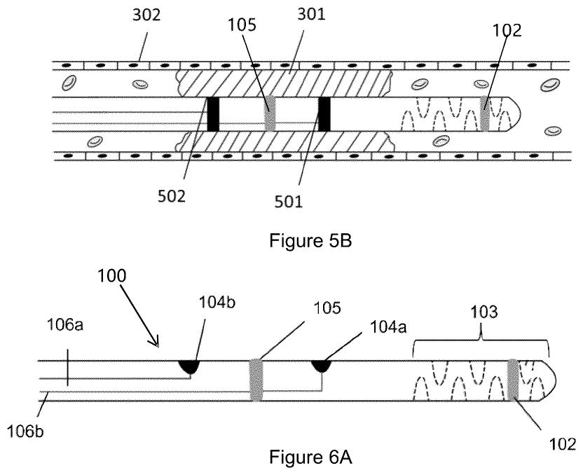

With reference to FIGS. 5A and 5B, the guide 100 in accordance with the present disclosure works similarly to prior art solid core guide wires; however, guide wire 100 includes the novel additional diagnostic function used to detect/determine one or more properties of thrombus 301 in the vasculature. When guide wire 100 reaches the thrombus site, the guide wire is inserted through thrombus 301. The impedance of thrombus 301 can optionally be recorded at various sections across the length of a portion or the entire length of the thrombus 301 as the guide wire is moved through thrombus 301 to allow for multiple points of measurement. Such a measurement method can be used to provide a better representation of the entire thrombus composition as compared to measuring at a single point; however, it can be appreciated that only a single site measurement of thrombus 301 can be used to detect/determine one or more properties of thrombus 301 in the vasculature.

Pre-surgery equipment preparation of guide wire 100 includes attaching impedance analyzer wires 202a to impedance analyzer 204 using a standard connector or other type of connector. Impedance analyzer wires 202a can then be attached to free end 201 of electrical wires 106a, 106b by optional use of wire connector 203. During the procedure, guide wire 100 can be inserted into the patient's blood vessel 302 using a standard guide wire insertion method. FIGS. 5A and 5B illustrate guide wire 100 deployed inside blood vessel 302 where two sensors 104a, 104b lay across the stenosis. Prior to, during, or after guide wire 100 is inserted into thrombus 301, impedance analyzer 204 can be switched on. Guide wire 100 is pushed through thrombus 301 slowly to allow impedance measurements of thrombus 301 at various points along the length of thrombus 301. Upon contact of thrombus 301 with sensors 104a, 104b on guide wire 100, the impedance of thrombus 301 can be measured. When the physician has collected sufficient data about thrombus 301 to determine the suitable mechanical thrombectomy method, impedance analyzer 204 can be switched off. Thereafter, wire connector 203 can be optionally opened to release electrical wires 106a, 106b from wire connector 203 before proceeding with a mechanical thrombectomy procedure to treat thrombus 301.

FIG. 5B illustrates an alternative configuration of guide wire 100 that has been deployed inside blood vessel 302. Guide wire 100 includes two sensors 501, 502 that lay across thrombus 301. The two sensors are formed as bands that substantially or fully encircle the body of guide wire 100. The shape of the one or more sensors is non-limiting. As illustrated in FIG. 5B, the sensor has a shape of a ringed band. As illustrated in FIG. 5A, sensors 104a, 104b have a square or rectangular shape that does not substantially encircle the outer surface of the guide wire.

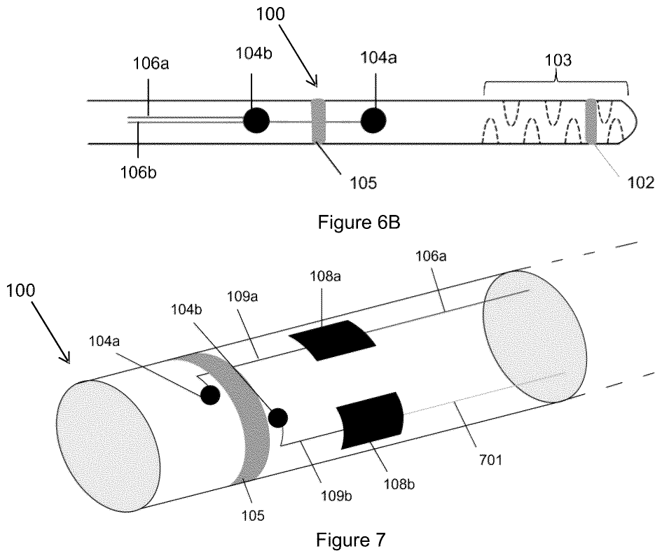

FIGS. 6A, 6B, and 7 are alternative configurations of the electrical wire design on guide wire 100. FIGS. 6A and 6B illustrate an embodiment of diagnostic guide wire 100 without solid core support wire 107. If the pushability of guide wire 100 is sufficient for it to reach the thrombus site without the need for a support wire 107, solid core support wire 107 may be removed. Such removal increases the lumen space for additional sensors on guide wire 100, which allows for more surface coverage of the guide wire with sensors, thus a more comprehensive analysis of thrombus 301 can be obtained.

In contrast to FIG. 1A, FIG. 7 illustrates an isometric view of one electrical wire (e.g., 106b) replaced by a solid core support wire 701 inside diagnostic guide wire 100. Solid core support wire 701 can optionally have a dual function. Support wire 701 can be used to conduct electrical signals to/from the one or more sensors and/or provide extra support to the guide wire (e.g., function similarly to solid wire support wire 107) as compared to the normal electrical wire 106b in guide wire 100. Solid core support wire 701 can have a greater density, greater cross-section area, and/or greater rigidity than the material used to form electrical wire 106a, 106b.

Referring now to FIGS. 8, 9A, 9B, there is illustrated two non-limiting variations of the diagnostic device application in accordance with the present disclosure. Instead of using a guide wire 100, the sensors may be placed on a stent retriever, an inflatable balloon, or a catheter.

Referring now to FIG. 8, there is provided a stent retriever 804. The operation and use of stent retriever is known in the art, thus will not be described herein. The stent retriever is illustrated as including a plurality of connected wires 805 that are connected together and the distal end is connected to a retrieval wire 806 that is used to move the stent retriever in the vascular. The stent retriever is illustrated as including a plurality of sensors. In one non-limiting configuration, the stent retriever includes four sensors, namely 802a, 802b, 802c and 802d. As can be appreciated, the stent retriever can include more than four sensors or less than four sensors (e.g., 2-20 sensors and all values and ranges therebetween). The location of the sensors on the stent retriever is non-limiting. As illustrated in FIG. 8, the sensors are spaced apart from one another and also one or more or all of the sensors are spaced from the ends of the stent retriever.

The stent retriever is also illustrated as including one or more optional distal marker bands 801a, 801b, 801c that are located in the distal tip or distal region of the stent retriever. The one of more distal markers are used to locate the stent retriever in the vascular during angiography. The materials used to form the distal mark bands can be the same or similar to the material used to form marker band 105 as discussed above. As illustrated in FIG. 8, additional sensor marker bands 803a, 803b, 803c, 803d can be optionally placed closely adjacent (e.g., less than 20 μm) from one or more of sensors 802a, 802b, 802c, 802d to facilitate in identifying the sensor location in the vascular during angiography. The location and number of sensors 802a, 802b, 802c, 802d, sensor marker bands 803a, 803b, 803c, 803d, and marker bands 801a, 801b, 801c vasculature is not limited to the configuration illustrated in FIG. 8. The configuration of the stent receiver illustrated in FIG. 8 can be used to identify the thrombus 301, and lesion and tissue composition and properties.

Referring now to FIGS. 9A and 9B, there is illustrated a balloon guide wire 906 that includes a plurality of sensors 903a, 903b on the balloon guide wire. The features such as the optional distal marker band 901, optional distal tip cuts 902, optional sensor marker band 904, and electrical wire 905a, 905b are similar in function to the marker bands 102, 105, distal cuts and gaps 103, wires 106a, 106b of guide wire 100 illustrated in FIGS. 1A and 1B, thus details of these features will not be repeated herein.

FIGS. 9A and 9B illustrate balloon guide wire 906 that that has a two sensors 903a, 903b mounted on a flexible substrate 908, thus allowing sensors 903a, 903b to come in direct contact with thrombus 301 when the flexible substrate is partially or fully expanded (e.g., inflated, etc.), or prior to expansion. The two sensors 903a, 903b illustrated in FIG. 9A are located on the outer surface of the flexible substrate 908. The number and location of sensors 903a, 903b and one or more marker bands 901, 904 are not limited to the sites shown in FIGS. 9A and 9B. Generally, two or more sensors are located on the flexible substrate 908. One or more mark bands, when used, can be located at or near the proximal and/or distal end of the flexible substrate 908, and/or located on the flexible substrate 908. As illustrated in FIGS. 9-1 and 9-2, marker band 904 is located on the flexible substrate 908 and between sensors 903a, 903b. The location of sensors 903a, 903b and one or more marker bands are not limited to the sites shown in FIGS. 9A, 9B [symbol] . The configuration of the balloon guide wire illustrated in FIGS. 9A, 9B can be used to identify the thrombus 301, and lesion and tissue composition and properties.

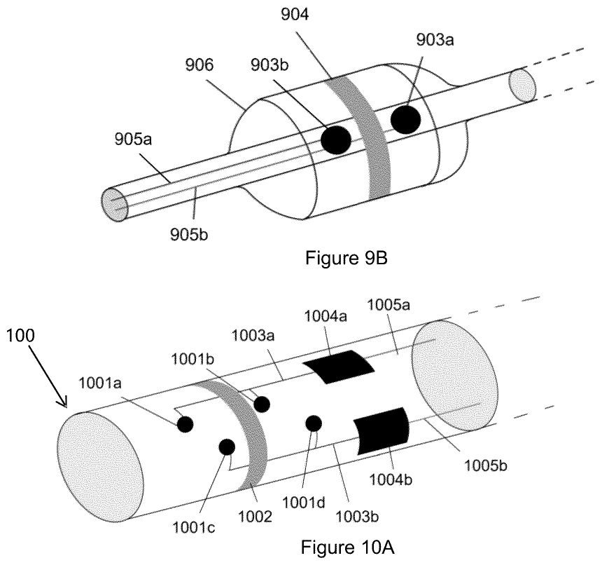

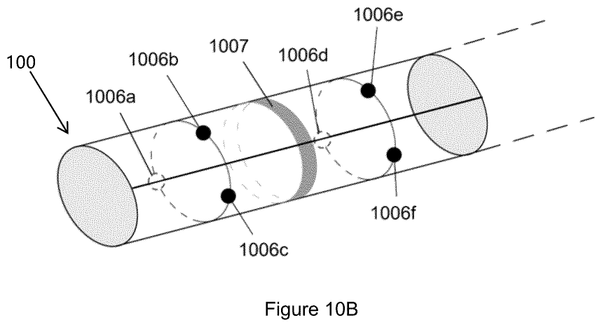

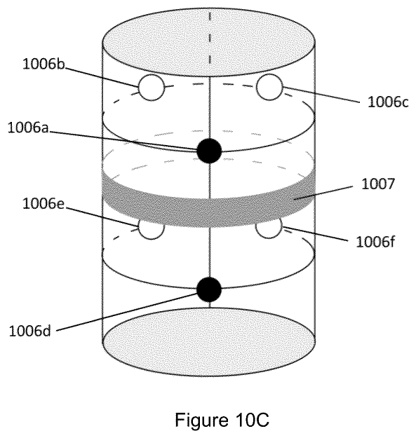

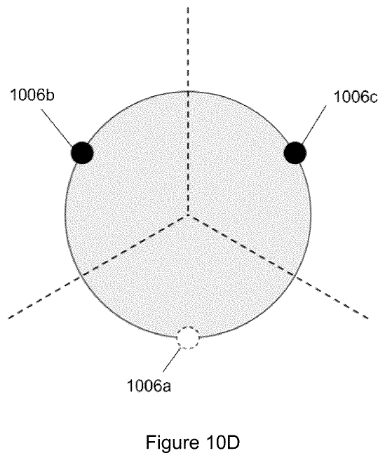

Referring now to FIGS. 10A, 10B, 10C, 10D, there is illustrated additional embodiments of multiple-sensors used on a guide wire 100. FIG. 10A illustrates an alternative design using four sensors 1001a, 1001b, 1001c, 1001d on guide wire 100. FIGS. 10B, 10C illustrate alternative designs using six sensors 1006a, 1006b, 1006c, 1006d, 1006e, 1006f on guide wire 100. FIGS. 10C and 10D illustrate the side and horizontal cross-sectional view of a non-limiting sensor placement in a six-sensor configuration. As can be appreciated, other numbers of sensors on guide wire 100 or on other types of devices discussed above (e.g., stent retriever, balloon guide wire, etc.) can be used. Also, the orientation of the sensors on guide wire 100 or other types of devices discussed above is non-limiting. As can be appreciated, when the number of sensors is increased on guide wire 100 or other types of devices discussed above, the number of electrical wires and/or solid core support wire 701 may also be increased; however, this is not required. As illustrated in FIG. 10A, one set of sensors 1001a and 1001b are connected to wire 1003a, and sensors 1001c and 1001d are connected to wire 1003b. In this arrangement one or more sensors are connected to one wire and one or more other sensors are connected to another wire, such that one set of sensors is connected to a single wire that extends to the distal end of the guide wire and another set of sensor is connected to another single wire that extends to the distal end of the guide wire.

The features such as the sensor marker bands 1002, 1007, conductive metals 1003a, 1003b, contact pads 1004a, 1004b, and electrical wires 1005a, 1005b are similar in function to the marker bands 102, 105, distal cuts and gaps 103, wires 106a, 106b of guide wire 100 illustrated in FIGS. 1A and 1B, thus details of these features will not be repeated herein.

When more than two sensors are used on the guide wire, the sensors can be used to cover more surface of guide wire 100 or other types of devices discussed above, and such additional information from use of the additional sensors can be used to potentially shorten procedure time as physicians may not require multiple points of measurement to obtain the overall impedance of thrombus 301.

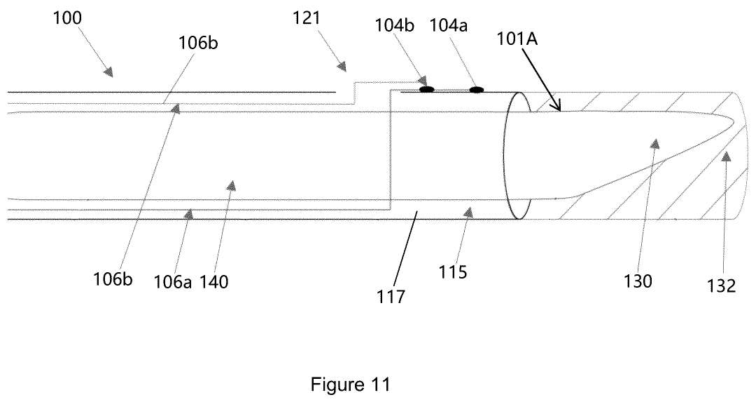

FIG. 11 illustrates other features that can be used on the guide wire 100. The guide wire of FIG. 11 is similar to the guide wire illustrated in FIGS. 1A and 1B, thus similar features of the guide wire will not be repeated herein. The outer surface of the tube 115 includes an opening 121 that is configured to allow the wire from the sensors to pass into the cavity of the tube. The location of the opening 121 on the tube is non-limitations. As illustrated in FIG. 11, the opening 121 is spaced form the sensors; however, it can be appreciated that opening 121 can be positioned beneath the one or more sensors.

A solid core 140 is illustrated as being positioned in the cavity 117 of the tube 115. The solid core 140 is an alternative to core support wire 107 as illustrated in FIG. 1A. The solid core 140 can be used to replace on of wires 106a or 106b; however, this is not required. In such an arrangement, the solid core is formed of an electrically conductive material. As illustrated in FIG. 11, the wires in the cavity of the tube are located between the outer surface of the core support wire 107 and the inner surface of the tube. The core can optionally be secured to one or more locations in the cavity of the tube; however, this is not required. In one non-limiting arrangement, 1-99.9% (and all values and ranges therebetween) of the longitudinal length of tapered distal tip 130 are not connected to the cavity of the tube. As illustrated in FIG. 11, the cross-sectional area of solid core 140 is larger than the cross-sectional area of core support wire 107 as illustrated in FIG. 1A. In one non-limiting embodiment, the cross-sectional area of solid core 140 is 20-95% (and all values and ranges therebetween) of the cross-sectional area of the cavity of the tube. Solid core 140 can be configured to extend to the distal end of the tube; however, this is not require. Generally, the material used to form solid core 140 can be the same or different from the material used to form the tube.

Distal section 101A of the guide wire includes a tapered distal tip 130. The shape of tapered distal tip 130 is non-limiting (e.g., conical shaped, cone shaped, pyramid shaped, single tapered side or ramped shape, etc.). Tapered distal tip 130 can be used to facilitate in the movement and/or insertion of guide wire 100 through the vascular system of a patient.

As illustrated in FIG. 11, the proximal end region of solid core 140 forms tapered distal tip 130; however, this is not required. The tapered distal tip can be formed by molding, grinding, shaving, etching, etc., distal section 101A of the guide wire. Alternatively, tapered distal tip 130 can optionally be formed of a flexible material (e.g., polymer, etc.) that has been molded or otherwise formed. Such a polymer distal tip can be connected to the end portion of the guide wire by use of an adhesive, melted connection, clamp, etc. The length and shape of tapered distal tip 130 is non-limiting. Generally, the length of tapered distal tip 130 is 0.05-8 cm (and all values and ranges therebetween). The tapered distal tip can optionally include a coating 132 that can be used in the movement and/or insertion of guide wire 100 through the vascular system of a patient. The coating can be a polymer material and/or a metal material. Generally the coating is formed of a different material that is used to form the tapered distal tip. Coating 132 (when used) can optionally include a radiopaque material to facilitate in monitoring and/or locating the distal portion of guide wire 100 as the guide wire is moved through the vascular system of a patient. The thickness of the coating is non-limiting. Generally, the coating thickness is less than 2 mm (e.g., 1 μm to 2 mm and all values and ranges therebetween). Alternatively, the optional radiopaque material can be in the form of a coil about the distal portion of the guide wire, and/or be a plated or coated material on the distal portion of the guide wire. As can be appreciated, the coil (when used) need not be formed of a radiopaque material. As can be appreciated, the use of a tapered distal tip 130, optional coating 132, and the optional radiopaque material can be used on any of the guide wires described above.

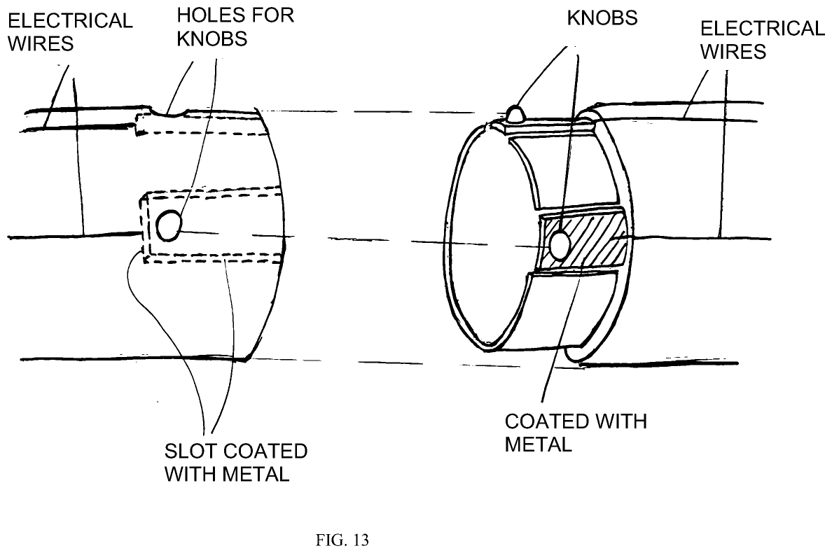

Referring now to FIG. 13, there is illustrated an optional attachment mechanism for connecting the wires (e.g. wires 106a, 106b, 112, etc.) to wire connector 203 or other type of wire connector. The electrical wires are optionally connected to the mating portions of the attachment mechanism which are optionally coated with metal. This is to ensure a good connection to supply power and allow the sensors to collect data. The attachment mechanism includes two mating locations positioned in a way to prevent the physician from attaching the parts incorrectly; however, it can be appreciated that other types and numbers of mating connections can be used. The knobs on the mating connections help secure the parts together, with the slots restricting any rotational movement applied during the procedure.

In various embodiments disclosed herein, a single component can be replaced by multiple components and multiple components can be replaced by a single component to perform a given function or functions. Except where such substitution would not be operative, such substitution is within the intended scope of the embodiments.

Additional features and methods of operation of the practice putting device are included in the figures.

A number of implementations have been described. Nevertheless, it will be understood that various modifications may be made without departing from the spirit and scope of the claims. Accordingly, other implementations are within the scope of the following claims.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

To aid the Patent Office and any readers of this application and any resulting patent in interpreting the claims appended hereto, Applicant does not intend any of the appended claims or claim elements to invoke 35 U.S.C. 112(f) unless the words “means for” or “step for” are explicitly used in the particular claim.

What is claimed:

1. A medical diagnostic system comprising:
a medical device that is configured to be inserted into a blood vessel of an animal or human; said medical device includes a distal portion having a distal end and a proximal portion having a proximal end;
an electrical assembly at least partially located at said distal portion of said medical device; said electrical assembly includes first and second sensors located at said distal portion of said medical device; said electrical assembly configured to transmit and receive one or more electrical signals at a diagnostic site when said distal portion of said medical device is located at the diagnosis site; and
an impedance analyzer configured to analyze one or more said signals received from said electrical assembly.

2. The medical diagnostic system as defined in claim 1, wherein said medical device is selected from the group consisting of a guide wire, a balloon guide wire, stent retriever or a catheter.

3. The medical diagnostic system as defined in claim 1, wherein said medical device includes a radiopaque marker positioned a) adjacent to one of said first or second sensors, b) between said first and second sensor, or c) distally to said first and second sensors.

4. The medical diagnostic system as defined in claim 1, wherein said electrical assembly includes a noise reduction sensor positioned on said distal portion of said medical device; said noise reduction sensor spaced from said first and second sensors; said noise reduction sensor positioned a) proximally to one of said first and second sensors, b) between said first and second sensors, or c) distally to one of said first and second sensors.

5. The medical diagnostic system as defined in claim 1, further includes an electronic circuit selected from the group consisting of a differential amplifier, and an amplifier noise canceller.

6. The medical diagnostic system as defined in claim 1, The medical diagnostic system as defined in claim 1, wherein said first sensor and/or second sensor are exposed an outer surface of said medical device so that at least a portion of said first and/or second sensors can contact an inner wall of a blood vessel or material located on the inner wall of the blood vessel when said distal portion of said medical device is positioned the diagnosis site.

7. The medical diagnostic system as defined in claim 6, wherein said medical device includes a tube; an outer surface of said tube includes first and second sensor recesses; said first sensor recess configured to at least partially receive said first sensor; said second sensor recess configured to at least partially receive said second sensor; a top surface of said first sensor is flush with or recessed from an outer surface of said tube when said first sensor is positioned in said first sensor recess; a top surface of said second sensor is flush with or recessed from an outer surface of said tube when said second sensor is positioned in said second sensor recess.

8. The medical diagnostic system as defined in claim 1, wherein said medical device includes a tube that includes an internal cavity' said internal cavity extends at least 3% of a longitudinal length of said tube; said electrical assembly including a first wire electrically connected to said first sensor and a second wire electrically connected to said second sensor; said first and second wires positioned in at least 10% of a longitudinal length of said cavity of said tube.

9. The medical diagnostic system as defined in claim 6, wherein said medical device includes a tube that includes an internal cavity' said internal cavity extends at least 3% of a longitudinal length of said tube; said electrical assembly including a first wire electrically connected to said first sensor and a second wire electrically connected to said second sensor; said first and second wires positioned in at least 10% of a longitudinal length of said cavity of said tube.

10. The medical diagnostic system as defined in claim 8, wherein a majority of a longitudinal length said first and second wires that are located in said cavity are not connected to said cavity.

11. The medical diagnostic system as defined in claim 1, wherein a distal portion of said medical device includes one or more recesses or cut-out portions that are configured to increase a flexibility of said distal portion of said medical device; said one or more recesses or cut-out portions positioned a) proximally to said first and second sensors, b) between said first and second sensors, or c) distally to said first and second sensors.

12. The medical diagnostic system as defined in claim 8, wherein said medical device includes a rod that is positioned in said cavity of said tube and extends from a proximal end of said tube to a distal portion of said tube; a majority of a length of said rod is not connected to said cavity.

13. The medical diagnostic system as defined in claim 1, wherein said medical device includes a tube substrate; said first and second sensors are connected to a top surface of said tube substrate.

14. The medical diagnostic system as defined in claim 13, wherein said tube substrate includes a shaved region that reduces a cross-sectional area of said shaved region as compared to regions of said tube substrate located proximal and/or distal to said shaved region; said first and second sensors at least partially positioned on said shaved region.

15. The medical diagnostic system as defined in claim 13, wherein said tube substrate has a longitudinal length of 1-25% a longitudinal length of a body or tube of said medical device; said tube substrate has a cross-sectional shaped that is the same or similar to a) a cross-sectional shape of said cavity of said tube, or b) a void region in said body.

16. The medical diagnostic system as defined in claim 13, wherein said medical device includes a body or tube that includes a cut-out area which positioned at a distal portion of said body or tube; said tube substrate positioned in said cavity and oriented related to said cut-out area such that a top surface of said first and second sensors that are connected to said top surface of said tube substrate are exposed an outer surface of said medical device.

17. The medical diagnostic system as defined in claim 2, wherein said medical device is a balloon guide wire; said balloon guide wire includes a tube and an inflatable balloon; an outer surface of said balloon includes said first and second sensors.

18. The medical diagnostic system as defined in claim 2, wherein said medical device is a stent retriever; said stent retriever includes a plurality of interconnected wires; said first and second sensors connected to an outer surface of said interconnected wires.

19. The medical diagnostic system as defined in claim 1, wherein a distal portion of said medical device includes a tapered region.

20. The medical diagnostic system as defined in claim 19, wherein said tapered region of said distal portion is at least partially coated with a radiopaque material.

21. A method for using said medical diagnostic system as defined in claim 1 to a) obtain one or more properties of a thrombus in a blood vessel, and/or b) identify an existence or presence of cancer cells flowing within the blood vessel.

22. A medical device that is configured to be inserted into a blood vessel of an animal or human; said medical device includes a tube and an electrical assembly; said tube includes an internal cavity that extends at least 3% of a longitudinal length of said tube; said tube has a distal portion that has a distal end; said tube has a proximal portion that has a proximal end; said electrical assembly at least partially located at said distal portion of said tube; said electrical assembly includes first and second sensors; said electrical assembly includes a first wire electrically connected to said first sensor and a second wire electrically connected to said second sensor; said first and second wires positioned in at least 10% of a longitudinal length of said cavity of said tube; said first and second sensors are located at said distal portion of said tube; said electrical assembly configured to transmit and receive one or more electrical signals at a diagnostic site when said distal portion of said tube is located at the diagnosis site.

23. The medical device as defined in claim 22, further including a radiopaque marker positioned a) adjacent to one of said first or second sensors, b) between said first and second sensor, or c) distally to said first and second sensors.

24. The medical device as defined in claim 22, wherein said electrical assembly includes a noise reduction sensor positioned on said distal portion of said tube; said noise reduction sensor spaced from said first and second sensors; said noise reduction sensor positioned a) proximally to said first and second sensors, b) between said first and second sensors, or c) distally to said first and second sensors.

25. The medical device as defined in claim 22, wherein said first or second sensors are exposed an outer surface of said tube so that at least a portion of said first or second sensors can contact an inner wall of a blood vessel or material located on the inner wall of the blood vessel when said distal portion of said tube is positioned the diagnosis site.

26. The medical device as defined in claim 22, wherein an outer surface of said tube includes first and second sensor recesses; said first sensor recess configured to at least partially receive said first sensor; said second sensor recess configured to at least partially receive said second sensor; a top surface of said first sensor is flush with or recessed from an outer surface of said tube when said first sensor is positioned in said first sensor recess; a top surface of said second sensor is flush with or recessed from an outer surface of said tube when said second sensor is positioned in said second sensor recess.

27. The medical device as defined in claim 22, wherein a majority of a longitudinal length said first and second wires that are located in said cavity are not connected to said cavity.

28. The medical device as defined in claim 22, wherein a distal portion of said tube includes one or more recesses or cut-out portions that are configured to increase a flexibility of said distal portion of said tube; said one or more recesses or cut-out portions positioned a) proximally to said first and second sensors, b) between said first and second sensors, or c) distally to said first and second sensors.

29. The medical device as defined in claim 22, further including a rod that is positioned in said cavity of said tube and extends from a proximal end of said tube to a distal portion of said tube; a majority of a length of said rod is not connected to said cavity.

30. The medical device as defined in claim 22, further including a tube substrate that is positioned at least partially in said cavity of said tube; said first and second sensors are connected to a top surface of said tube substrate; said tube substrate formed of a different material from said tube.

31. The medical device as defined in claim 30, wherein said tube substrate includes a shaved region that reduces a cross-sectional area of said shaved region as comparted to regions of said tube substrate located proximal and/or distal to said shaved region; said first and second sensors at least partially positioned on said shaved region.

32. The medical device as defined in claim 30, wherein said tube substrate has a longitudinal length of 1-25% a longitudinal length of said tube; said tube substrate has a cross-sectional shaped that is the same or similar to a cross-sectional shape of said cavity of said tube.

33. The medical device as defined in claim 30, wherein said tube includes a cut-out area positioned at the distal portion of said tube; said tube substrate positioned in said cavity and oriented relative to said cut-out area such that a top surface of said first and second sensors that are connected to said top surface of said tube substrate are exposed an outer surface of said medical device.

34. The medical device as defined in claim 22, wherein a distal portion of said tube includes a tapered region.

35. The medical device as defined in claim 34, wherein said tapered region is coated with a radiopaque material.

36. A method for using said medical device as defined in claim 22 to a) obtain one or more properties of a thrombus in a blood vessel, and/or b) identify an existence or presence of cancer cells flowing within the blood vessel.
